(19) United States
(12) Patent Application Publication
(10) Pub. No.: US 2024/0361751 A1
(43) Pub. Date: Oct. 31, 2024

(54) METHOD FOR PROVIDING CONTROL DATA FOR A LASER OF A PROCESSING APPARATUS, CONTROL DEVICE, COMPUTER PROGRAM AND COMPUTER-READABLE MEDIUM

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/645,797

(22) Filed: Apr. 25, 2024

(30) Foreign Application Priority Data
Apr. 28, 2023 (DE) 10 2023 111 157.4

(51) Int. Cl. G05B 19/4155

(52) U.S. Cl. CPC G05B 19/4155; G05B 2219/45165

(57) ABSTRACT

A method for providing control data for a laser of a processing apparatus, wherein the method includes the following steps performed by at least one control device: outputting control data to a processing apparatus, wherein the control data effect that laser pulses are sequentially output onto positions of impingement into a processing area to be processed along an incision path by the laser of the processing apparatus. The method further includes following steps: ascertaining an effective diameter of a local effective area generated by the respective laser pulse, in the respective position of impingement, ascertaining a unit area to be formed by a pulse distance and a row distance depending on the effective diameter and an energy dose to be provided in the processing area, ascertaining the pulse distance and row distance according to a preset ascertaining method, generating the control data for controlling the processing apparatus.

### FIELD

The present invention relates to a method for providing control data for a laser of a processing apparatus. In addition, the invention relates to a control device, to a processing apparatus, to a computer program, and to a computer-readable medium.

### BACKGROUND

Processing apparatuses for laser-based processing an object are known from the prior art and are for example employed in the surface processing of objects of metal. A laser-based material processing can be effected in objects of optically transparent materials by means of inducing an optical breakdown in the material. For inducing the optical breakdown, energy can be provided by laser pulses at a predetermined place, until the formation of a cavitation bubble occurs at the place. It effects separating or cutting the material. The laser-based processing can also be effected by means of LIRIC/RIS (refractive index shaping) or CXL (cross linking) besides effecting optical breakdowns. Herein, it is not necessarily required that a threshold for effecting the optical breakdown is reached. Instead, it can be required to exceed another preset threshold or to reach a preset range of values in the last mentioned methods. In this case, it can also be required to ensure a preset spatial distribution of the positions of impingement of the laser pulses and/or a preset temporal distribution of points of time of the impingement of the laser pulses.

A further field of application of the processing apparatus for laser-based processing is the medical technology. In the medical technology, the laser-based processing apparatuses are applied as processing apparatus for example for correcting an optical visual disorder and/or pathologically or unnaturally altered areas of the cornea. Therein, a pulsed laser and a beam focusing device can for example be formed such that laser pulses effect a photodisruption and/or ablation due to the optical breakdown in a focus situated within an organic tissue, to remove a tissue, in particular a tissue lenticule, from the cornea. Besides a separation of the tissue, processing the tissue can also be generally provided for example to change a local structure of the tissue by laser pulses.

Widespread methods for laser treatment of the cornea like the photodisruption and/or the ablation are based on specifically inducing the optical breakdown in the corneal tissue by the pulsed laser. The optical breakdown describes a strong local ionization of the corneal tissue, wherein a critical plasma density is exceeded. The optical breakdown can be initiated in the corneal tissue in laser-induced manner in that a power density threshold is exceeded by the laser pulse within the corneal tissue. For this purpose, the laser pulse is focused to a focusing point in the corneal tissue, at which it then has a maximum power density, which exceeds the power density threshold. Upon exceeding the critical plasma density, the local absorbance of the corneal tissue increases, whereby the plasma temperature is severely increased. Due to the temperature increase of the plasma, a Coulomb expansion of the plasma occurs, whereby a cavitation bubble arises in the corneal tissue, in which the corneal tissue is severed. By focusing laser pulses to the predetermined focusing points, it is thereby possible to sever the corneal tissue in the focusing points in specific and locally restricted manner. The photodisruption is based on a local mechanical decomposition of the corneal tissue, which is caused by a shock wave arising in the optical breakdown.

For separating the cornea on a certain processing area to be processed, it is required to guide the laser pulses to positions of impingement within the processing area to be processed by the laser of a processing apparatus. The cavitation bubbles form on the positions of impingement, which have a bubble diameter in the processing area to be processed, at which the tissue is locally separated.

According to the prior art, the laser pulses are sequentially guided to the respective positions of impingement by the processing apparatus. Therein, the positions of impingement are arranged along a preset incision path within the processing area to be processed. The course of the incision path forms a two-dimensional or three-dimensional pattern in the processing area to be processed, which is usually developed as a raster pattern or meander pattern. The pattern can also be formed as a helical pattern, spiral pattern or volute pattern. The positions of impingement are usually symmetrically arranged in the processing area to be processed. This means that pulse distances between adjacent positions of impingement along the incision path and pulse distances between adjacent positions of impingement transverse to the incision path or distances of adjacent rows of the incision path are identical. Due to the identical distances of the positions of impingement along both directions, the patterns formed by the incision are also referred to as symmetric patterns.

The pulse distances between adjacent positions of impingement along the incision path can describe the distances between adjacent positions of impingement, which are oriented along a row of the incision path. The path direction along the incision path is also referred to as fast direction. Accordingly, the pulse distances along the incision path are referred to as fast pulse distances.

The distances of adjacent rows of the incision path in a position of impingement transverse to the incision path can describe the distances between adjacent rows along the transverse direction in the positions of impingement. The transverse direction transverse to the incision path is also referred to as slow direction. Accordingly, the pulse distances transverse to the incision path are referred to as slow pulse distances.

For processing, for example for separating the tissue in the processing area to be processed, it is required to select the pulse distances between adjacent positions of impingement such that the bubbles of adjacent positions of impingement contact or overlap each other. However, an energy locally supplied by the pulse energies of the laser pulses in a contiguous period of time is to be kept as low as possible. The same applies to an entire supplied energy into the processing area to be processed. This is attributable to the fact that local tissue defects, in particular opaque bubble layers, can otherwise form.

### SUMMARY

The invention is based on the object to optimize incision paths to a reduction of the introduced energy.

This object is solved by the method according to the invention, the control device according to the invention, the processing apparatus according to the invention, the computer program according to the invention as well as the computer-readable medium according to the invention. Advantageous configurations with convenient developments of the invention are specified in the respective dependent claims, wherein advantageous configurations of the method are to be regarded as advantageous configurations of the processing apparatus, of the control device, of the computer program and of the computer-readable medium and vice versa.

A first aspect of the invention relates to a method for providing control data for a processing apparatus.

The control data can include a respective dataset for outputting laser pulses in the cornea. Additionally or alternatively, a respective dataset for adjusting at least one beam device for beam guidance and/or beam shaping and/or beam deflection and/or beam focusing of a laser beam of the respective laser can be included in the control data.

The method includes the following steps performed by at least one control device.

The method includes outputting the control data to a processing apparatus, wherein the control data effects that laser pulses are sequentially output to positions of impingement into a processing area to be processed along an incision path by the laser of the processing apparatus. The processing area can be three-dimensionally defined. In a simple case, it can be a two-dimensional processing area on a surface of the object. Therein, the incision path can two-dimensionally extend within the processing area. Herein, the path direction and the transverse direction can also be situated within the two-dimensional processing area. The processing area can also be three-dimensionally defined and for example enclose a volume within the object. In this case, the processing area can for example be defined by points, the coordinates of which can also include a depth in relation to a distance to the surface. In this case, the incision path can also three-dimensionally extend within the processing area. In this case, the path direction and the transverse direction can also be three-dimensionally defined.

In other words, control data is generated and communicated to the processing apparatus by the control device, which instructs the processing apparatus to output the laser pulses by the laser of the processing apparatus. The control data is configured such that it causes the sequential output of the laser pulses onto positions of impingement into the processing area to be processed by the processing apparatus. The output of the laser pulses onto the positions of impingement can be effected in two manners. The control data can include concrete coordinates of the positions of impingement along the incision path such that the laser pulses are output onto the preset coordinates. Another variant provides that only pulse distances between the respective positions of impingement and row distances between the rows of the incision path are preset by the control data. In this variant, the laser pulses can be output by a laser of the processing apparatus during a movement of a focusing point with a preset frequency, therein, the pulse distance of the positions of impingement along the incision path depends on the frequency. Therein, the pulse distance along the incision path decreases with the higher frequency. Accurate coordinates of the positions of impingement are not preset in this variant. A row distance of the rows of the incision path transverse to the incision path can be adjusted by the course of the incision path.

Therein, the control data presets pulse distances to be complied with of the positions of impingement of the laser pulses to directly adjacent positions of impingement. It can be provided that the positions of impingement are defined by their pulse distances to the adjacent positions of impingement. The sequential output of the laser pulses onto the respective positions of impingement is effected along the incision path. The directly adjacent positions of impingement have the pulse distance to each other along a path direction of the incision path. The directly adjacent rows have the row distance to each other along a transverse direction of the incision path. In other words, the positions of impingement are spaced from each other with the pulse distance along a main direction of the incision path, which is also referred to as path direction of the incision path. The pulse distance can be measured from a center of a position of impingement of one of the laser pulses to a center of a position of impingement of a further laser pulse along the path direction. The pulse distance can also be referred to as spot distance in the literature. Therein, the pulse distances can relate to the laser pulses, which are output onto the positions of impingement along the incision path for processing the processing area to be processed. In contrast, possible parasitic laser pulses, subthreshold laser pulses or laser pulses, which can be output according to a possible other method, are not understood as concerned laser pulses in terms of the application.

Along a secondary direction of the incision path, which is also referred to as transverse direction of the incision path, adjacent rows are spaced from each other with the row distance. The row distance can also be referred to as track distance in the literature.

For example, it can be provided that the laser pulses are sequentially emitted along the incision path in a raster into the processing area to be processed. The emission of the laser pulses can be effected such that they are applied along respective rows of the processing area to be processed. The path direction can be oriented along the respective rows. After applying the laser pulses along the respective row, application of the laser pulses in a following one of the rows can be effected, which can be arranged parallel to and along the transverse direction to the preceding row.

For example, the transverse direction can be oriented transversely, in particular perpendicularly, to the path direction in the direction of the adjacent rows. For example, the transverse direction can describe a column direction of the incision path in the raster. Within the respective row, the positions of impingement can be spaced from each other by the pulse distances. Between adjacent rows, the positions of impingement can be spaced from each other by the row distances along the transverse direction.

The transverse direction and the path direction can be identically oriented across the pattern or they can be locally differently oriented across the pattern. The local different orientation can for example be present if the incision path has a helical line, volute, spiral or any course. Therein, the path direction describes the course along the path and the transverse direction the course transverse to the path.

It is provided that the method comprises the following further steps.

In a step, ascertaining an effective diameter of a local effective area generated by the respective laser pulse of the pulse energy to be adjusted in the respective position of impingement within the processing area is effected. Instead of a pulse energy to be adjusted, it can also be a preset, but known pulse energy. In other words, it is ascertained by the control device, which effective diameter the respective local effective area has within the processing area in the position of impingement, which is induced by the respective laser pulse comprising the pulse energy to be adjusted. In particular, the effective diameter can describe a diameter of a cavitation bubble formed in the respective position of impingement. By ascertaining the effective diameter, an extent of a local effect of the laser pulse, for example of a local separation of the tissue in the respective positions of impingement can be ascertained. The effective diameter can also be different from the diameter of the cavitation bubble formed in the respective position of impingement. The effective diameter can also have a preset ratio to the diameter of the cavitation bubble formed in the respective position of impingement. The effective diameter can also describe a diameter related to the ascertained effective area, independently of the accurate shape of the effective area. For example, the effective diameter R can be calculated by a formula R=K*(pulse energy−threshold) \hat{ }(⅓), wherein K can be a preset or adjustable factor. The threshold can for example describe an energy, from which a cavitation bubble forms. The local effective area can also describe the area, with which a material or tissue is changed according to the intended processing within the processing area.

In a further step, ascertaining a unit area to be formed by the pulse distance and the row distance depending on the effective diameter and a preset energy dose to be provided or known is effected. The unit area can have an identical surface area as the local effective area or a surface area deviating therefrom. In other words, the unit area is ascertained by the control device, which is to be provided by the pulse distance and the row distance. The unit area can preset a product of the pulse distance and the row distance of a position of impingement. The preset energy dose to be provided can be preset in the control device, be ascertained by the control device or be transferred to the control device. The energy dose can for example be defined by pulse energy/unit area or in alternative form by pulse energy/(pulse distance*row distance).

The pulse energy to be adjusted can for example be ascertained based on the known energy dose to be adjusted. The energy dose to be adjusted can for example be ascertained based on the known pulse energy to be adjusted.

In a following step of the method, ascertaining the pulse distance as well as ascertaining the row distance depending on the unit area according to a predetermined ascertaining method is effected. The ascertaining method can include an application of a preset formula, a preset simulation or retrieving the pulse distance as well as the row distance from a look-up table.

In a following step, the control data for controlling the processing apparatus is generated. For example, the control data can include the pulse distance, the row distance and/or the respective positions of impingement of the respective laser pulses. For example, the control data can preset a course of the row distance and a course of the pulse distance along a route of the incision path. This specification can allow ascertaining the course of the incision path. The course of the incision path can also be ascertained according to the row distance and added to the control data. The pulse distance can be described along the length of the incision path.

The invention also includes developments, by which additional advantages arise.

A development of the invention provides that the local effective area generated in the respective position of impingement is ascertained depending on a local separation depth of the position of impingement in the processing area to be processed. In other words, it is considered by the control device in ascertaining the respectively generated local effective area, in which separation depth the respective position of impingement is situated. For example, it can be provided that the processing area is within a material, wherein the separation depth can describe a distance of the respective position of impingement to a surface of the material. The distance can describe a path length to be traveled by the laser pulse. The control device can consider an absorption coefficient of the material stored in the control device to ascertain an energy of the laser pulse absorbed by the material before reaching the position of impingement.

A development of the invention provides that the pulse distance and the row distance have an asymmetric ratio to each other. In other words, the pulse distance and the row distance have different values. The pulse distance and the row distance have the ratio to each other, which is unequal to 1 to 1.

A development of the invention provides that the pulse distance and the row distance have the ratio between 10:9 inclusive and 10:1 inclusive, preferably between 5:4 inclusive and 2:1 inclusive, to each other. In other words, the pulse distance is larger than the row distance, wherein the ratio between the pulse distance and the row distance is between 10 to 9 inclusive and 10 to 1 inclusive. Preferably, the ratio between the pulse distance and the row distance is between 5 to 4 inclusive and 2 to 1 inclusive. The ratio can also be between 9:8 inclusive and 9:1 inclusive

A development of the invention provides that the pulse distance and the row distance have the ratio between 9 to 10 inclusive and 1 to 10 inclusive, preferably between 4:5 inclusive and 1:2 inclusive, to each other. In other words, the row distance is larger than the pulse distance, wherein the ratio between the pulse distance and the row distance is between 9 to 10 inclusive and 1 to 10 inclusive. Preferably, the ratio between the pulse distance and the row distance is between 4 to 5 inclusive and 1 to 2 inclusive. The ratio can also be between 8:9 inclusive and 1:9 inclusive.

A development of the invention provides that the preset ascertaining method includes the following further steps. One of the further steps includes ascertaining the pulse distance according to a preset optimization method, wherein the optimization method is configured to parameterize the pulse distance such that a local energy density along the path direction is minimized. A next one of the further steps includes ascertaining the row distance from the unit area and the pulse distance.

In other words, ascertaining the pulse distance as well as ascertaining the row distance are effected according to the preset optimization method. The optimization method is adapted such that the pulse distance is parameterized such that the local energy density has a minimum local energy density along the respective path direction. In other words, the optimization method, for example a mathematical minimization method, is applied by the control device, which is adapted to optimize the pulse distance to the effect that the energy input by the laser pulses along the path direction results in the minimum local energy density along the path direction. For example, it can be provided that the pulse distance is optimized such that the local energy is input by the laser pulses with respect to the path direction along the path direction of the incision path, for example within a respective row of the incision path, which is minimized. The local energy can for example describe a sum of the pulse energies of the laser pulses, which are arranged within the respective row.

Since the unit area to be formed and the pulse distance are ascertained, the row distance is ascertained from the unit area to be formed and the pulse distance. For example, this can be effected by a division of the unit area to be formed by the pulse distance.

A development of the invention provides that at least one boundary condition is preset in the optimization method that the pulse distance is less than or equal to or smaller than the effective diameter. In other words, at least the boundary condition is preset to the optimization method that the pulse distance has a value, which is smaller than or less than or equal to the effective diameter.

A development of the invention provides that at least one boundary condition is preset in the optimization method that the pulse distance is greater than or equal to or larger than the effective diameter. In other words, at least the boundary condition is preset to the optimization method that the pulse distance has a value, which is larger than or greater than or equal to the effective diameter.

A development of the invention provides that at least one boundary condition is preset in the optimization method that the local power density along the path direction satisfies a preset local power density condition. The power density condition can for example preset a lower and/or an upper threshold value, which is to be complied with by the power density. Thereby, it can be ensured that a minimum power density for igniting the plasma is complied with and/or a too high power density, which can result in defects, is prevented.

A development of the invention provides that ascertaining the pulse distance as well as the row distance according to a preset ascertaining method depending on the unit area includes the following steps. Ascertaining the pulse distance depending on a pulse distance specification received by the control device. In other words, the pulse distance specification is received by the control device, which can for example preset an admissible range of values of the pulse distance. The pulse distance specification can for example be provided by a user interface. A further one of the steps includes ascertaining the row distance depending on the pulse distance and the unit area, wherein the row distance results from a division of the unit area by the pulse distance. In other words, the unit area is divided by the pulse distance to ascertain the row distance.

A development of the invention provides that ascertaining the pulse distance as well as the row distance according to a preset ascertaining method depending on the unit area includes the following steps. Ascertaining the row distance depending on a row distance specification received by the control device. In other words, the row distance specification is received by the control device, which can for example preset an admissible range of values of the row distance. For example, the row distance specification can be provided by a user interface. A further one of the steps includes ascertaining the pulse distance depending on the row distance and the unit area, wherein the pulse distance results from a division of the unit area by the row distance. In other words, the unit area is divided by the row distance to ascertain the pulse distance.

A development of the invention provides that includes the following further steps. Retrieving a preset range of values of an admissible pulse energy of the laser pulses for processing the processing area including at least a lower threshold value of the admissible pulse energy, ascertaining the pulse energy to be adjusted of the respective laser pulses depending on the lower threshold value of the admissible pulse energy according to a preset relation. In other words, receiving a preset range of values of an admissible pulse energy of the laser pulses for processing the processing area is effected. The preset range of values includes at least a lower threshold value of the admissible pulse energy, which the respective laser pulses are allowed to have. In other words, the preset range of values is preset to the control device, which describes the admissible pulse energy, which the respective laser pulses are allowed to have. The preset range of values can for example depend on a type of a material and/or a tissue in the processing area to be processed. The range of values has at least the lower threshold value. In other words, at least a lower limit of the pulse energy of the respective laser pulses is preset by the range of values. In particular, the lower threshold value can describe a threshold value, which can be required for forming a local plasma for separating the tissue by a respective laser pulse.

In a step, the pulse energy to be adjusted of the respective laser pulses is ascertained depending on the lower threshold value of the admissible pulse energy according to a preset relation by the control device. In other words, the pulse energy to be adjusted of the respective laser pulses depends on the lower threshold value of the admissible pulse energy. The pulse energy to be adjusted is ascertained by the control device according to the preset relation depending on the lower threshold value of the admissible pulse energy. The preset relation can for example include a formula. The preset relation can for example include a multiplication by a preset factor.

A development of the invention provides that the preset relation presets a factor between 1.25 inclusive and 4 inclusive, preferably a factor between 1.5 inclusive and 3 inclusive, even more preferably a factor of 2.2, with respect to the lower threshold value. In other words, the pulse energy to be provided is calculated from the minimum energy by means of the preset relation. Therein, the pulse energy to be provided is multiplied by the factor, which is between 1.25 and 4, preferably between 1.5 and 3, or is 2.2, to ascertain the pulse energy to be adjusted.

A development of the invention provides that the method includes a further step, which includes comparing the minimum local energy dose to an admissible local energy dose range. In other words, it is provided that the ascertained minimum local energy dose is compared to the admissible local energy dose range by the control device. The method includes a step of increasing the pulse energy by a predetermined correction value upon falling below the admissible local energy dose range and reducing the pulse energy by the predetermined correction value upon exceeding the admissible local energy dose range. In other words, it is examined by the control device if the minimum local energy dose is within the admissible local energy dose range. In case that the minimum local energy dose is below the admissible local energy dose range, the pulse energy is increased by the predetermined correction value. In case that the minimum local energy dose is above the admissible local energy dose range, the pulse energy of the respective laser pulses is reduced by the predetermined correction value. By the development, the advantage arises that complying with a preset admissible local energy dose range can be ensured.

Furthermore, the method for controlling the processing apparatus also includes the step of transferring the provided control data to at least one ophthalmological laser of the processing apparatus.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A second aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

The control device is configured for providing control data for a laser of a processing apparatus. The control device is configured to output the control data to a processing apparatus, wherein the control data effect that laser pulses are sequentially output onto positions of impingement into a processing area to be processed along an incision path by the laser of the processing apparatus, wherein the positions of impingement have a pulse distance to each other along a path direction of the incision path and the positions of impingement have a row distance to each other along a transverse direction of the incision path.

It is provided that the control device is configured to receive a preset range of values of an admissible pulse energy of the laser pulses for processing the processing area. The preset range of values of the admissible pulse energy of the laser pulses includes at least a lower threshold value of the admissible pulse energy. The control device is configured to ascertain a pulse energy to be adjusted of the respective laser pulses depending on the lower threshold value of the admissible pulse energy according to a preset relation. The control device is configured to ascertain an effective diameter of a local effective area generated by the respective laser pulse, which has the pulse energy to be adjusted, in the respective position of impingement. The control device is configured to ascertain a unit area to be formed by the pulse distance and the row distance depending on the effective diameter. It is provided that the control device is configured to ascertain the pulse distance as well as the row distance according to an optimization method. The control device is configured to ascertain the pulse distance as well as the row distance such that a local energy dose along one of the directions has a minimum local energy dose. In the optimization method, at least the boundary condition is preset that the pulse distance and the row distance are smaller than the pulse diameter. The control device is configured to generate the control data for controlling the processing apparatus.

A third aspect of the invention relates to a processing apparatus with at least one laser and at least one control device, which is formed to perform the steps of at least one embodiment of one of the previously described methods according to the first aspect. The respective laser can be formed to sequentially output laser pulses, which have the pulse energy to be adjusted, onto positions of impingement.

A fourth aspect of the invention relates to a computer program. The computer program includes commands, which for example form a program code. The program code can include at least one control dataset with the respective control data for the control device. Upon execution of the program code by means of a computer or a computer cluster, it is caused to execute the previously described method according to the first aspect of the invention or at least one embodiment thereof.

A fifth aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the developments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figures. The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a processing apparatus with a laser, according to an embodiment.

FIG. 2 depicts a schematic representation of an arrangement of positions of impingement along an incision path, according to an embodiment.

FIG. 3 depicts a schematic representation of a further arrangement of positions of impingement along an incision path, according to an embodiment.

FIG. 4 depicts a schematic representation of a further arrangement of positions of impingement along an incision path, according to an embodiment.

FIG. 5 depicts a schematic representation of a further arrangement of positions of impingement along an incision path, according to an embodiment.

FIG. 6 depicts a schematic representation of a sequence of a method, according to an embodiment.

In the figures, identical or functionally identical elements are provided with the same reference characters.

### DETAILED DESCRIPTION

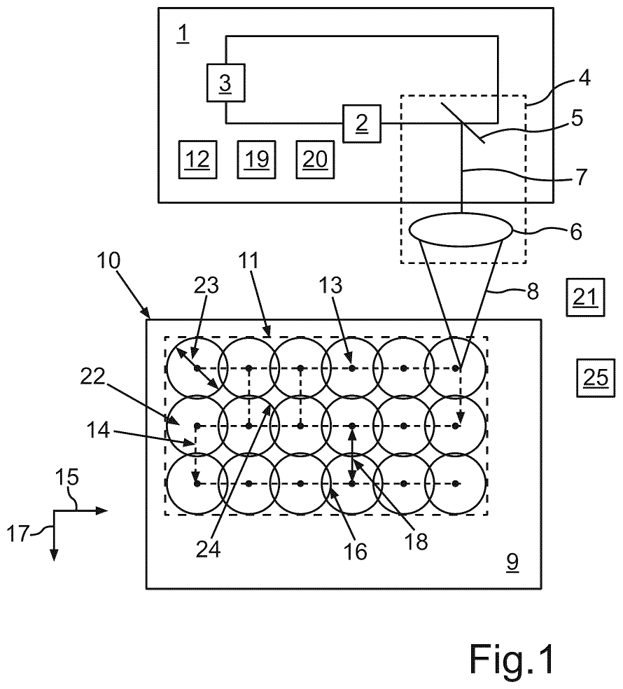

FIG. 1 shows a schematic representation of a processing apparatus with a laser.

A schematic representation of a processing apparatus 1 with a laser 2 for a separation of a material 9 of an object 10 in a predefined processing area 11 is shown. The predefined processing area 11 can be arranged on a surface of the object 10 or in a predetermined separation depth with respect to the surface of the object 10. For separating the material 9 in the processing area 11, the laser 2 can be configured for outputting laser pulses 7 onto positions of impingement 13, which can have a pulse energy 21 to be adjusted. A photodisruption of the material 9 in the position of impingement 13 can be induced by the laser pulses 7, whereby the material 9 can be locally separated in the position of impingement 13.

One recognizes that a control device 3 for the laser 2 can be formed besides the laser 2. The control device 3 can be configured to generate control data 12 and to communicate it to the treatment apparatus. The control data 12 can be configured to instruct the processing apparatus 1 for outputting the laser pulses 7 onto the preset positions of impingement 13. The control data 12 can instruct the processing apparatus 1 for providing the laser pulses 7 with a pulse energy 21 to be adjusted. The processing apparatus 1 can comprise the control device 3. Alternatively thereto, the control device 3 can be a component external with respect to the processing apparatus 1.

Furthermore, FIG. 1 shows that the laser pulse 7 generated by the laser 2 can be deflected towards a processing area 11 of a material 9 to be processed by means of a beam device 4, which can include a beam deflection device 5 such as for example a rotation scanner. The beam deflection device 5 is also controlled by the control device 3 to for example generate interfaces, preferably also incisions or cuts, by separating the material 9 on the processing area 11 to be processed. The laser pulse 7 can be guided by a focusing device 6, which can adjust the beam path 8 of the laser pulse 7 for focusing the laser pulse 7.

Preferably, the illustrated laser 2 can be a photodisruptive laser 2, which is formed to emit the laser pulses 7 in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. Optionally, the control device 3 additionally comprises a storage device, not illustrated, for at least temporary storage of at least one control dataset, wherein the control dataset or datasets include(s) control data 12 for positioning and/or for focusing individual laser pulses 7 in the material 9. The positions of impingement 13 of the individual laser pulses 7 can be preset by the control data 12.

The control device 3 can be configured to generate the control data 12 and to send it to the processing apparatus 1. The control data 12 can instruct the processing apparatus 1 to separate the processing area 11 to be processed by outputting the laser pulses 7 into the processing area 11 to be processed. The processing of the object 10 in the processing area 11 to be processed can be effected by outputting the laser pulses 7 onto the positions of impingement 13 within the processing area 11 to be processed. The laser pulses 7 can effect local removal of material 9 in the respective positions of impingement 13. In the respective position of impingement 13, a cavitation bubble or generally a local effective area 22 can for example be formed, which has an effective diameter 23 in the processing area 11 to be processed, in which the material 9 is removed or decomposed. In order to allow processing the material 9, the control device 3 can be configured to preset the distances between positions of impingement 13 in the processing area 11 to be processed such that the local effective areas 22 of the positions of impingement 13 together process the processing area 11 to be processed.

In the figures, the positions of impingement 13 of the laser pulses 7 are arranged in a regular pattern for better comprehension and for better representation. However, it is a special case. Correspondingly, the unit area 24 is also illustrated in simplified manner to allow a better comprehension.

The positions of impingement 13 can be arranged along an incision path 5, which can be ascertained by the control device 3. The processing apparatus 1 can be configured to sequentially output the laser pulses 7 along the incision path 5 onto the respective positions of impingement 13. The incision path 5 can for example be formed as a spiral course, as a raster course or as a meander course. For effecting the local processing of the material 9 in a position of impingement 13, it can be required that a minimum energy is to be provided by the respective laser pulse 7 in the position of impingement 13. The predetermined minimum energy can be preset as a lower threshold value 20 of an admissible range of values 19 of the pulse energy 21. The admissible range of values 19 can for example be preset in the control device 3, ascertained by the control device 3, for example depending on a material 9 of the object 10, or be received by the control device 3. The control device 3 can be configured to ascertain a pulse energy 21 to be adjusted of the laser pulses 7 from the lower threshold value 20 of the admissible pulse energy 21 by means of a predetermined relation. The preset relation can for example preset that the pulse energy 21 to be adjusted of the laser pulses 7 is 1.5 times inclusive to 3 times inclusive, in particular 2.2 times, the lower threshold value 20.

The effective diameter 23, which describes the local effective area 22 in the respective position of impingement 13 of a laser pulse 7, can depend on the respective pulse energy 21 to be adjusted of the laser pulse 7. The control device 3 can be configured to ascertain the effective diameter 23 in the respective position of impingement 13 depending on the pulse energy 21 to be adjusted. For example, the ascertainment can be effected by applying a model, a formula, a simulation or a look-up table, wherein the effective diameter 23 can be assigned to the pulse energy 21 to be adjusted. The effective diameter 23 can also depend on a local material 9 in the respective position of impingement 13, therein, a material dependency can also be considered by the control device 3.

For example, the incision path 14 can have rows arranged parallel to each other. The individual rows can be spaced from each other by row distances.

For processing the processing area 11, it can be required to ascertain a pulse distance 16, which can describe a distance of the respective positions of impingement 13 along a path direction 15 of the incision path 5. In addition, it can be required to ascertain a row distance 18, which can describe a distance of adjacent positions of impingement 13, which can be arranged transversely to the incision path 14, for example along a transverse direction 17 of the incision path 5. The pulse distance as well as the row distance 18 can be ascertained by the control device 3 depending on the effective diameter 23. The pulse distance as well as the row distance 18 can be dimensioned such that the tissue is processed, for example separated, as desired within the processing area 11. For ascertaining the pulse distance 16 as well as the row distance 18, the control device 3 can be configured to ascertain a unit area 24 to be formed by the pulse distance 16 and the row distance 18 depending on the effective diameter 23. The unit area 24 to be formed can be provided to provide an energy dose 25 to be provided. The energy dose 25 to be provided can describe an average energy per processing area 11, which is to be provided by the laser pulses 7 in the processing area 11 to be processed.

In a further step, the control device 3 can be configured to apply an optimization method to ascertain the pulse distance 16. For example, the optimization method can be a minimization method, which can be configured to minimize a local energy dose 25 along the path direction 15 of the incision path 5. In other words, the optimization method is provided to ascertain the pulse distance 16, which is required to allow processing of the processing area 11 and results in the minimum energy dose 25 in the path direction 15 of the incision path 5 at the same time. The local energy dose 25 along the path direction 15 of the incision path 5 can describe the energy per area or line element, which is provided to the processing area 11 to be processed along the path direction 15 by the laser pulses 7.

As the boundary condition of the optimization method, it can be preset that the pulse distance 16 has at least the value of the effective diameter 23. Preferably, it can be provided that the minimization of the local energy dose 25 is provided along the path direction 15. This can be advantageous because an input local power into the tissue is thereby minimized at the same time. Thereby, a formation of defects such as for example an opaque bubble layer is prevented or at least made more improbable.

It can be provided that the pulse energies 21 of the laser pulses 7 have to be adapted in order that the local energy dose 25 is within a preset dose range. For example, it can be provided that the local energy dose 25 has to be between a certain minimum value and a certain maximum value of the local energy dose 25.

In case that the local energy dose 25 is outside of the range of values 19, the control device 3 can be configured to increase or to decrease the pulse energy 21 to be adjusted of the respective laser pulses 7 by a preset value.

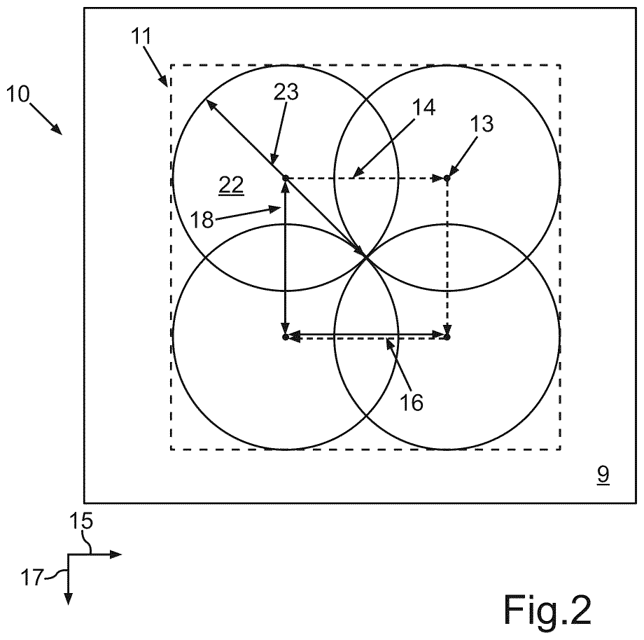

FIG. 2 shows a schematic representation of an arrangement of positions of impingement 13 along an incision path 5.

The positions of impingement 13 can have a local effective area 22, which can have the effective diameter 23. The positions of impingement 13 can be arranged symmetrically to each other. By a symmetric arrangement, it can to be understood that the pulse distance 16 and the row distance 18 have identical values. The row distance 18 and the pulse distance 16 can be smaller than the effective diameter 23 such that the local effective areas 22 can overlap each other. The row distance 18 and the pulse distance 16 could be selected such that the local effective areas 22 of the positions of impingement 13 have an identical overlap factor of 1.41 along the path direction 15 as well as along the transverse direction 17.

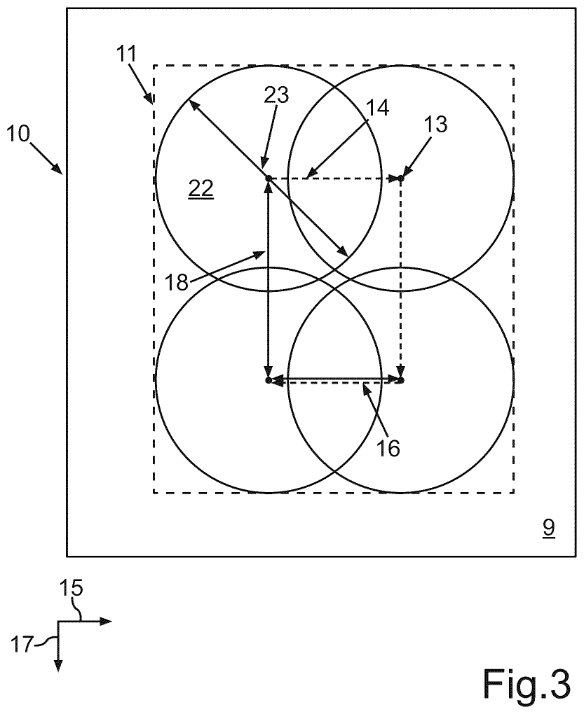

FIG. 3 shows a schematic representation of a further arrangement of positions of impingement 13 along an incision path 5.

FIG. 3 shows a schematic representation of an asymmetric arrangement of positions of impingement 13. The pulse distances 16 can have lower values than the row distances 18. Thereby, the positions of impingement 13 can be arranged more densely to each other along the path direction 15 than along the transverse direction 17. Due to the denser arrangement of the positions of impingement 13 along the path direction 15, a higher local energy dose 25 is supplied to the row of the incision path 5 along the path direction 15 than along the transverse direction 17.

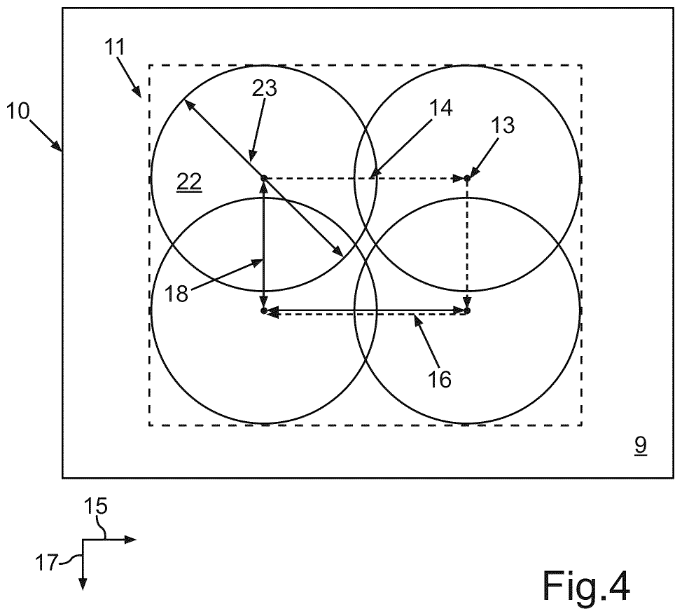

FIG. 4 shows a schematic representation of a further arrangement of positions of impingement 13 along an incision path 5.

FIG. 4 shows a schematic representation of a further asymmetric arrangement of the positions of impingement 13. The shown arrangement can also be referred to as inversely asymmetric. The row distances 18 can have lower values than the pulse distances 16. Thereby, the positions of impingement 13 can be arranged more densely to each other along the transverse direction 17 than along the path direction 15. Due to the denser arrangement of the positions of impingement 13 along the transverse direction 17, a higher local energy dose 25 is supplied to the column of the incision path 5 along the transverse direction 17 than along the path direction 15. The shown arrangement of the positions of impingement 13 is particularly advantageous in a sequential application of the laser pulses 7 along the incision path 5. This is attributable to the fact that the local energy dose 25 is lower along the path direction 15 in the shown arrangement than in the arrangements, which are shown in FIG. 2 and FIG. 3. This results in the fact that the power, which is supplied along the path direction 15, is also lower. The lower power results in a risk of formation of defects being lower.

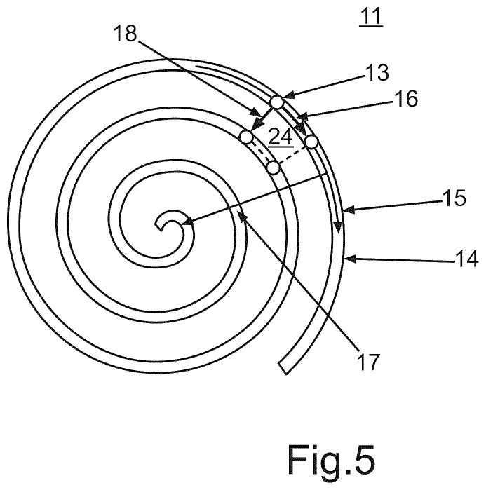

FIG. 5 shows a schematic representation of an arrangement of positions of impingement 13 along an incision path 14.

The shown incision path 14 can have a spiral course. Thereby, the orientation of the path direction 15 and of the transverse direction 17 can be locally different. In other words, the path direction 15 and the transverse direction 17 cannot be identical across the entire processing area 11, but depend on the local orientation of the incision path 14. The pulse distance 16 and the row distance 18 can locally vary to be able to locally provide the unit area 24.

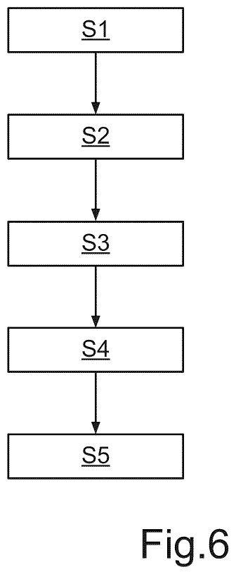

FIG. 6 shows a schematic representation of a sequence of a method.

The method can be performed by the control device 3, which is shown in FIG. 1. The method can be provided for providing control data 12 for a laser 2 of a processing apparatus 1. The method can be able to comprise the following steps performed by the control device 3.

Receiving a preset range of values 19 of an admissible pulse energy 21 of the laser pulses 7 for processing the processing area 11 including at least a lower threshold value 20 of the admissible pulse energy 21 in step S1.

Ascertaining a pulse energy 21 to be adjusted of the respective laser pulses 7 depending on the lower threshold value 20 of the admissible pulse energy 21 according to a preset relation in step S2.

Ascertaining an effective diameter 23 of a local effective area 22 generated by the respective laser pulse 7, of the pulse energy 21 to be adjusted, in the respective position of impingement 13 in step S3.

Ascertaining a unit area 24 to be formed by the pulse distance 16 and the row distance 18 depending on the effective diameter 23 and an energy dose 25 to be provided in step S4,

Ascertaining the pulse distance 16 as well as the row distance 18 according to a preset ascertaining method depending on the unit area 24 in step S5;

The method may further include generating the control data 12 for controlling the processing apparatus 1.

The method may further include outputting control data 12 to a processing apparatus 1, wherein the control data 12 effect that laser pulses 7 are sequentially output onto positions of impingement 13 into a processing area 11 to be processed along an incision path 5 by the laser 2 of the processing apparatus 1, wherein the positions of impingement 13 have a pulse distance 16 to each other along a path direction 15 of the incision path 5 and the positions of impingement 13 have a row distance 18 to each other along a transverse direction 17 of the incision path 5. The control data 12 can preset the course of the incision path 14. The control data 12 can for example preset distances between rows of the incision path 14 by the row distance 18. The control data 12 can preset the pulse distances 16 between the positions of impingement 13. This can for example be effected by presetting a temporal and/or local output frequency of the laser pulses.

Usually, there are three different types of arrangements of the positions of impingement 13. For applying the laser pulses 7 to the positions of impingement 13, the processing apparatus 1 guides a focus point along an incision path 5 across the processing area 11 to be processed. The incision path 14 can be formed as a raster, meander, spiral, circle or as another pattern.

For all of these incision paths 14, a direction “along the scan pathway” and a direction “across the scan pathway” applies. The former is also referred to as path direction 15, the latter as transverse direction 17.

The pulse distance 16 along the path direction 15 is usually also referred to as spot distance because it describes the distance between positions of impingement 13 of consecutive laser pulses 7. The row distance 18 along the transverse direction 17 can describe a distance between adjacent rows and is usually referred to as track distance because it describes the distance between two adjacent rows/tracks, on which the positions of impingement 13 are arranged.

It is spoken of a symmetric arrangement if the row distance 18 and the pulse distance 16 are exactly identical or identical within a certain tolerance range. Other arrangements are referred to as asymmetric arrangements. A type of the asymmetric arrangements describes such arrangements, in which the pulse distance 16 is smaller than the row distance 18. In this type, the positions of impingement 13 of the consecutively output laser pulses 7 are arranged more densely to each other, while the rows are arranged with a lower density to each other. The application of the positions of impingement 13 along the incision path 5 can be compared to plotting a line with a pen.

Another type of the asymmetric arrangements describes such ones, in which the row distance 18 is smaller than the pulse distance 16. In this type, the positions of impingement 13 of the consecutively output laser pulses 7 are arranged less densely to each other, while the rows are arranged with a higher density to each other. This type is also referred to as reverse or inverse asymmetric arrangement.

With an asymmetric arrangement of the positions of impingement 13, it can be operated with a lower energy per pulse. For processing the processing area 11 to be processed with a symmetric arrangement of the positions of impingement 13, it is to be ensured that the local effective areas 22 overlap each other in an X- and a Y-direction such that a contiguous processing area 11 is processed by the local effective areas 22 together.

With an asymmetric arrangement of the positions of impingement 13, it is only to be ensured that the local effective areas 22 overlap each other in the X- or the Y-direction. The overlap in the corresponding other direction is ensured by the preset density defined by the unit area 24. The differences between the pulse distances 16 and the row distances 18 can preferably be selected such that a difference is at least 25-30%, and up to 100%. Thus, the ratios can be between 5:4 and 2:1 or 4:5 and 1:2.

The ratios can also be between 10:9 and 10:1 or 9:10 and 1:10 or between 9:8 and 9:1 or 8:9 and 1:9.

The relation to the lower threshold value 20 is separate thereto. Below the lower threshold value 20 of the pulse energy 21, bubbles are not formed in the positions of impingement 13. In this case, the pulse energy 21 can for example only be absorbed and/or passed up to the retina. Only if a certain pulse energy 21 and a certain power density are reached, a plasma is formed, which then converts into a pressure wave. This pressure wave has an increased temperature and acts as a bubble, which locally severs the material 9. The power density describes an energy per processing area unit and time unit. A time of a laser pulse 7 can be in the range of femtoseconds.

Ascertainments of an optimum pulse energy 21 for a symmetric arrangement have yielded that it is 3 times the lower threshold value 20. In addition, the ascertainments have shown that the optimum pulse energy 21 is 1.5 times the lower threshold value 20. Further details to the ascertainment can be taken from the publication Arba-Mosquera, Samuel, et al. 2021 Arba-Mosquera, Samuel, et al. “Analytical optimization of the cutting efficiency for generic cavitation bubbles.” Biomedical Optics Express 12.7 2021:3819-3835.

For technical reasons, fluctuations of the pulse energy 21 of the laser pulses 7 can occur. In order to be able to compensate for them, the pulse energy 21 to be adjusted can be increased by 5%.

The inverse asymmetry has the advantage that a spatial distance between the positions of impingement 13 of the consecutive laser pulses 7 is increased. Thereby, less interactions are effected in the positions of impingement 13 of the consecutive laser pulses 7 along the path direction 15. The time interval between positions of impingement 13 of the laser pulses 7 along the transverse direction 17 is extended. The positions of impingement 13 of the laser pulses 7 along the transverse direction 17 result in a larger overlap of the local effective areas 22 in this arrangement. However, they are applied with an increased time interval to each other such that a lower interaction between the positions of impingement 13 occurs. Thereby, the laser pulses 7 effect a proper processing in the inverse asymmetry, in particular a proper separation in contrast to the direct asymmetry. In addition, the processing is even more proper than in the symmetric arrangement. This is attributable to the fact that the spatial and temporal course severely and significantly depends on the pulse energy 21.

The overlapping area of adjacent positions of impingement 13 results from a multiplication of the pulse distance 16 by the row distance 18. This area is also referred to as unit area 24. The unit area 24 determines the energy dose 25 per area.

For example, multiplications of different row distances 18 and fast pulse distances 16 16 result in a same unit area 24:

2.5 μm×5.0 μm=12.5 μm²; 5.0 μm×2.5 μm=12.5 μm²; and 3.5 μm×3.5 μm μm=12.3 μm².

In all three variants, the average spatial overlap and thus the applied energy dose 25 per processing area 11 are identical.

What changes is the asymmetry and thereby the local power.

At 2.5 μm×5.0 μm, the positions of impingement 13 of the consecutive laser pulses 7 come closer to each other. Thereby, a higher local power results along the path direction 15: at 5.0 μm×2.5 μm, the positions of impingement 13 of the consecutive laser pulses 7 are farther away from each other. Thereby, a lower local power results along the path direction 15. At 3.5 μm×3.5 μm, the power along the path direction 15 is between the two asymmetric cases.

The density of the positions of impingement 13 is identical in the three variants because the pulse distances 16 and the row distances 18 are each smaller than the effective diameter 23 in all three variants. If one neglects a time of the treatment, the three variants would be equally well suitable for processing the processing area 11. This is also the case according to the prior art.

However, therein, it is neglected that the time has a substantial influence on the processing of the processing area 11. However, the time is to be considered due to the dependency of the power on the time to be able to improve a quality of the processing.

A radius of a laser pulse 7 in cross-section is about 3 um and is independent of the pulse energy 21. A radius of a bubble formed in the position of impingement 13 of the laser pulse 7 is dependent on the pulse energy 21. For example, it is 2 μm at 85 nJ and 3 μm at 110 nJ and can for example be ascertained by simulations.

It can be provided to ascertain a suitable pulse distance 16 and a suitable row distance 18. A combination of a pulse distance 16 of 5 μm and of a row distance 18 of track 2.5 μm could be possible example values.

For the ascertained values, the pulse energy 21 to be adjusted of the laser pulses 7 can optionally be adapted. Upon a risk of formation of an opaque bubble layer, the pulse energy 21 to be adjusted can for example be reduced by a correction value of 5 nJ. Upon a difficult separation in the periphery, the pulse energy 21 can be increased by 5 nJ. Upon a risk of formation of black spots, which do not stem from dirt or debris, the pulse energy 21 can also be increased by 5 nJ.

Alternatively, it can also be provided that the energy dose 25 is for example preset instead of the pulse energy 21. For example, it can also be provided that the row distance 18, the pulse distance 16 as well as the energy dose 25 are preset. For these values, the required pulse energy 21 to be adjusted could then be ascertained. The pulse energy 21 to be adjusted can for example be ascertained based on the known energy dose 25 to be adjusted. The energy dose 25 to be adjusted can for example be ascertained based on the known pulse energy 21 to be adjusted.

Overall, the examples show how a processing of a processing area can be improved by an asymmetric arrangement of the positions of impingement.

1. A method for providing control data for a laser of a processing apparatus, wherein the method comprises the following steps performed by at least one control device:
outputting control data to a processing apparatus, wherein the control data effect that laser pulses are sequentially output onto positions of impingement into a processing area to be processed along an incision path by the laser of the processing apparatus, wherein the positions of impingement have a pulse distance to each other along a path direction of the incision path, and adjacent rows of the incision path have a row distance to each other along the incision path in a transverse direction of the incision path,
ascertaining an effective diameter of a local effective area generated by the respective laser pulse with a pulse energy to be adjusted in the respective position of impingement,
ascertaining a unit area to be formed by the pulse distance and the row distance depending on the effective diameter and an energy dose to be provided in the processing area,
ascertaining the pulse distance as well as the row distance of the incision path according to a preset ascertaining method depending on the unit area, and
generating the control data for controlling the processing apparatus (1).

2. The method according to claim 1, wherein the local effective area generated in the respective position of impingement is ascertained depending on a local processing depth of the processing area to be processed.

3. The method according to claim 1, wherein the pulse distance and the row distance have an asymmetric ratio to each other.

4. The method according to claim 1, wherein the pulse distance and the row distance have an asymmetric ratio to each other, wherein the pulse distance and the row distance have a ratio between 10:9 inclusive and 10:1 inclusive, preferably between 5:4 inclusive and 2:1 inclusive.

5. The method according to claim 1, wherein the pulse distance and the row distance have an asymmetric ratio to each other, wherein the pulse distance and the row distance have a ratio between 9:10 inclusive and 1:10 inclusive, preferably between 4:5 inclusive and 1:2 inclusive.

6. The method according to claim 1, wherein the preset ascertaining method comprises:
ascertaining the pulse distance according to a preset optimization method, wherein the optimization method is configured to parameterize the pulse distance such that a local energy density of an energy input into the processing area by the pulse energy of the respective laser pulses is minimized along the path direction, and
ascertaining the row distance (18) from the unit area and the pulse distance.

7. The method according to claim 1, wherein the preset ascertaining method comprises:
ascertaining the pulse distance according to a preset optimization method, wherein the optimization method is configured to parameterize the pulse distance such that a local energy density of an energy input into the processing area by the pulse energy of the respective laser pulses is minimized along the path direction, wherein the optimization method is configured to parameterize the pulse energy to be adjusted such that a local energy density is minimized along the path direction, and
ascertaining the row distance from the unit area and the pulse distance.

8. The method according to claim 1, wherein the preset ascertaining method comprises:
Ascertaining the pulse distance according to a preset optimization method, wherein the optimization method is configured to parameterize the pulse distance such that a local energy density of an energy input into the processing area by the pulse energy of the respective laser pulses is minimized along the path direction, wherein at least one boundary condition is preset in the optimization method that the pulse distance is greater than or equal to or larger than the effective diameter, and
ascertaining the row distance from the unit area and the pulse distance.

9. The method according to claim 1, wherein the preset ascertaining method comprises:
Ascertaining the pulse distance according to a preset optimization method, wherein the optimization method is configured to parameterize the pulse distance such that a local energy density of an energy input into the processing area by the pulse energy of the respective laser pulses is minimized along the path direction, wherein at least one boundary condition is preset in the optimization method that the pulse distance is greater than or equal to or larger than the effective diameter, and
ascertaining the row distance from the unit area and the pulse distance.

10. The method according to claim 1, wherein the preset ascertaining method comprises:
Ascertaining the pulse distance according to a preset optimization method, wherein the optimization method is configured to parameterize the pulse distance such that a local energy density of an energy input into the processing area by the pulse energy of the respective laser pulses is minimized along the path direction, wherein at least one boundary condition is preset in the optimization method that a local power density along the path direction satisfies a preset local power density condition, and
ascertaining the row distance from the unit area and the pulse distance.

11. The method according to claim 1, wherein ascertaining the pulse distance as well as the row distance according to a preset ascertaining method includes, depending on the unit area:
ascertaining the pulse distance depending on a pulse distance specification received by the control device, and
ascertaining the row distance depending on the pulse distance and the unit area, wherein the row distance results from a division of the unit area by the pulse distance.

12. The method according to claim 1, wherein ascertaining the pulse distance as well as the row distance according to a preset ascertaining method includes, depending on the unit area:
ascertaining the row distance depending on a row distance specification received by the control device, and
ascertaining the pulse distance depending on the row distance and the unit area, wherein the pulse distance results from a division of the unit area by the row distance.

13. The method according to claim 1, further comprising the following steps:
retrieving a preset range of values of an admissible pulse energy of the laser pulses for processing the processing area, including at least a lower threshold value of the admissible pulse energy, and
ascertaining a pulse energy to be adjusted of the respective laser pulses depending on the lower threshold value of the admissible pulse energy according to a preset relation.

14. The method according to claim 1, further comprising the following steps:
Retrieving a preset range of values of an admissible pulse energy of the laser pulses for processing the processing area, including at least a lower threshold value of the admissible pulse energy, and
ascertaining a pulse energy to be adjusted of the respective laser pulses depending on the lower threshold value of the admissible pulse energy according to a preset relation, wherein the preset relation has a value between 1.25 and 4, preferably of 2.2.

15. The method according to claim 1, further comprising the following steps:
comparing the minimum local energy dose to an admissible local energy dose range, p1 increasing the pulse energy to be adjusted by a predetermined correction value upon falling below the admissible local energy dose range, and
reducing the pulse energy to be adjusted by a predetermined correction value upon exceeding the admissible local energy dose range.

16. A control device , configured for providing control data for a laser of a processing apparatus, wherein the control device is configured
to output control data to a processing apparatus, wherein the control data effect that laser pulses are sequentially output onto positions of impingement into a processing area to be processed along an incision path by a laser of the processing apparatus, wherein the positions of impingement have a pulse distance to each other along a path direction of the incision path, and adjacent rows have a row distance to each other along a transverse direction of the incision path,
to ascertain an effective diameter of a local effective area generated by the respective laser pulse, of a pulse energy to be adjusted, in the respective position of impingement, to ascertain a unit area to be formed by the pulse distance and the row distance depending on the effective diameter and an energy dose to be provided in the area,
to ascertain the pulse distance as well as the row distance according to a preset ascertaining method depending on the unit area; and
to generate the control data for controlling the processing apparatus.

17. A processing apparatus with at least one laser for outputting laser pulses to positions of impingement in a processing area to be processed of an object , and at least one control device according to claim 16.

18. (canceled)

19. A computer-readable medium for storing a computer program, the computer program comprising commands, which cause a control device to execute the method according to claim 1.
